(19) United States
(12) Patent Application Publication
(10) Pub. No.: US 2026/0345060 A1
(43) Pub. Date: Nov. 5, 2026

(54) System and Method for Human Optimization via Subconscious Health Nudging (MOYTU Framework)

(71) Applicant: Doychin Karshovski, Loveland, OH (US)

(72) Inventor: Doychin Karshovski, Loveland, OH (US)

(21) Appl. No.: 19/655,106

(22) Filed: Apr. 22, 2026

Related U.S. Application Data
(60) Provisional application No. 63/799,720, filed on May 4, 2025.

(51) Int. Cl. A61B 5/375; A61B 5/00

(52) U.S. Cl. CPC A61B 5/375; A61B 5/7264

(57) ABSTRACT

A system and method for goal-directed behavioral optimization closes the gap between human intention and sustained behavioral action by delivering personalized sensory stimuli through any sensory, physiological, or neurological pathway—operating beneath the threshold of conscious resistance and calibrated to resist displacement by habitual avoidance, motivational depletion, or volitional self-override. Upon receiving a user-defined behavioral objective, the system enters a baseline observation period during which biometric, behavioral, contextual, and somatic data—including user-reported subjective somatic assessments not currently measurable by external sensing technologies—are collected to construct a personalized behavioral phenotype. The system then delivers a dual-stimulus architecture of reward and corrective cues, progressively reducing nudging frequency as behavioral alignment improves until the objective is embedded without external reinforcement. A closed adaptive feedback loop continuously refines stimulus parameters. The system is implementable via any information-processing architecture and applicable across individual, clinical, and institutional contexts.

### CROSS-REFERENCE TO RELATED APPLICATIONS

This application claims the benefit of priority under 35 U.S.C. § 119(e) to U.S. Provisional Patent Application No. 63/799,720, filed May 4, 2025, and entitled “System and Method for Human Optimization via Subconscious Health Nudging (MOYTU Framework),” the entire disclosure of which is incorporated herein by reference.

### FIELD OF THE INVENTION

The present invention relates to the fields of behavioral optimization systems, adaptive biofeedback technology, human performance engineering, and user-directed lifestyle design. More specifically, the invention provides a system and method whereby a user defines desired behavioral and lifestyle objectives, and the system delivers personalized stimuli through any sensory, physiological, or neurological pathway to progressively achieve those objectives—operating beneath the threshold of the user's active attentional filters and calibrated to resist displacement by habitual avoidance, motivational depletion, or volitional self-override. The invention is applicable across individual, clinical, and institutional contexts and is implementable via any information-processing architecture.

### BACKGROUND OF THE INVENTION

Despite significant advances in wearable technology, biometric sensing, and digital health platforms, a fundamental gap persists between human health knowledge and sustained behavioral action. Existing systems—including fitness trackers, wellness applications, behavioral coaching platforms, and biofeedback devices—share a common architectural limitation: they generate increasingly sophisticated data about the user's physiological and behavioral state, then present that data as conscious signals requiring human interpretation, decision, and volitional action.

This model contains multiple structural failure points. First, it places the full burden of behavior change on the conscious decision-making system—a resource that is finite, depletable under stress, and systematically outcompeted by the body's faster, older, sub-attentional preference and habit systems. Second, it assumes that better information produces better action—a premise that decades of commercial health technology have collectively failed to validate. Knowledge does not reliably produce action. The knowing-doing gap remains the central unsolved problem of human behavior change.

Third, and critically, all existing signal-based systems—including those that deliver stimuli directly to the body such as haptic vibration, audio prompts, or visual alerts—still require the user to consciously interpret the signal, make a decision, and take a volitional action in response. Even the most direct physical stimulus is rendered ineffective by this requirement, because it reintroduces the conscious mediation layer that habituation, decision fatigue, and motivational depletion routinely defeat.

Fourth, humans demonstrate a well-documented tendency to habituate to repeated signals regardless of their modality or intensity. Notifications are dismissed. Vibrations are ignored. Data dashboards go unread. Over time, even well-designed behavioral signals lose their efficacy as the conscious mind learns to filter them as background noise.

Fifth, no existing system provides a closed-loop reinforcement architecture capable of delivering both reward and corrective stimuli below the threshold of conscious resistance—leaving the critical behavioral reinforcement function dependent on the user's own volitional self-discipline, which is precisely the resource that behavior change systems are designed to compensate for.

No existing system addresses behavior change at the correct architectural level. All current approaches attempt to improve the quality, frequency, or presentation of information delivered to the conscious mind—without recognizing that the conscious mind is not the appropriate target for sustained behavioral intervention.

There exists a need for a system that bypasses conscious mediation entirely—delivering stimuli directly to the user's behavioral and physiological systems in a manner that promotes lasting alignment with user-defined objectives without requiring conscious interpretation, decision, or volitional effort. The present invention addresses this need.

### SUMMARY OF THE INVENTION

The present invention provides a system and method for closing the gap between human intention and human action—the most persistent and consequential failure point in behavioral science, personal health, and human performance. Setting a goal, without a system to execute it, is like writing a program and never pressing Enter. The intention exists. The desire is genuine. But nothing runs. The present invention—the MOYTU Framework—is the execution layer for human behavioral goals. It takes what a person has already decided they want and runs it silently, persistently, and without requiring continuous conscious participation to sustain it.

The MOYTU Framework operates through three sequential phases. In the first phase, the user defines one or more behavioral or lifestyle objectives—for example, improved sleep quality, increased physical movement, or reduced consumption of nutritionally depleted foods. These objectives may be freely defined by the user or selected from a pre-structured library aligned with the TEMELI Framework, a holistic behavioral health system developed by the inventor that organizes human optimization across six foundational domains: Breath, Sleep, Movement, Nutrition, Mindfulness, and Prevention. The TEMELI Framework provides Moytu with a scientifically grounded and philosophically coherent set of pre-validated behavioral targets, enabling users to select goals that are already known to produce measurable improvements in human health and performance.

In the second phase—the learning period—the system observes the user without intervening. It collects biometric, behavioral, contextual, and somatic data from wearable devices, environmental sensors, and user-reported inputs. It classifies observed behaviors as aligned or misaligned with the stated objective through three mechanisms: automated correlation analysis of collected data, user-initiated manual tagging, and retrieval from a pre-programmed behavioral library containing behaviors with established health outcome relationships. During this period, the system builds a personalized behavioral phenotype—a dynamic model of the user's unique patterns, rhythms, triggers, and responses. No stimuli are delivered during this phase. The system only listens.

In the third phase—the active nudging period—the system begins delivering low-friction stimuli calibrated to operate beneath the threshold of the user's active attentional filters. These stimuli employ a dual-stimulus architecture modeled on the fundamental principles of behavioral reinforcement: reward stimuli are delivered when the user engages in behaviors aligned with their stated objective, and corrective stimuli are delivered when behaviors misaligned with the objective are detected or predicted. This architecture—the carrot and the stick, calibrated to sub-attentional perception—creates a persistent, invisible reinforcement environment that progressively reshapes behavioral preferences without requiring the user's conscious cooperation or volitional effort to sustain.

Stimuli are delivered through any sensory, physiological, or neurological pathway available to the user, including but not limited to haptic signals, visual cues, auditory prompts, olfactory signals, thermal signals, or any modality, whether currently known or developed after the time of filing. The system is explicitly designed to resist the habituation effects that defeat all existing signal-based behavioral systems—by varying stimulus timing, modality, intensity, and emotional tone based on continuous feedback from measured physiological and behavioral responses.

As behavioral alignment with the stated objective improves, the system automatically reduces nudging frequency proportionally. The goal of the system is its own obsolescence in each behavioral domain—to embed new behaviors so deeply that external reinforcement is no longer required. The endpoint may be pre-programmed by the system or defined by the user.

The invention further incorporates an interoceptive intelligence layer—accepting user-reported subjective somatic assessments, including felt states of rightness and intuitive assessments of wellbeing that are not currently measurable by external sensing technologies, as first-class behavioral alignment data. This positions the user's own biological intelligence as a primary authority in the system—not merely a passive subject of optimization, but an active co-author of their own behavioral transformation.

Optionally, the system includes a suggestive priming module that pre-associates specific sensory stimuli with desired behavioral or emotional states through guided protocols including hypnosis, visualization, or meditation—enhancing the efficacy of subsequently delivered nudging stimuli by establishing sub-attentional associative pathways prior to the active nudging phase.

The invention is applicable across individual, clinical, and institutional contexts—including corporate wellness programs, military performance conditioning, clinical behavioral rehabilitation, and educational environments—and is implementable via any information-processing architecture, including digital computing systems, machine learning models, quantum computing systems, biological computing substrates, or any hybrid combination thereof.

In summary, the MOYTU Framework is a behavioral technology system uniquely designed to address the knowing-doing gap at its correct architectural level—through the novel combination of sub-attentional stimulus delivery, closed-loop behavioral reinforcement, somatic intelligence integration, and user-directed lifestyle design. Unlike motivational tools that rely on conscious engagement, the MOYTU Framework operates as a behavioral execution engine—implementing user-defined goals through sub-attentional stimulus delivery without requiring conscious participation to sustain.

### BRIEF DESCRIPTION OF THE DRAWINGS

FIG. 1 is a block diagram illustrating the system architecture of the MOYTU Framework, showing the goal input module, behavior classification module, learning module, nudging module, reduction module, and feedback loop, and the directional relationships between each module.

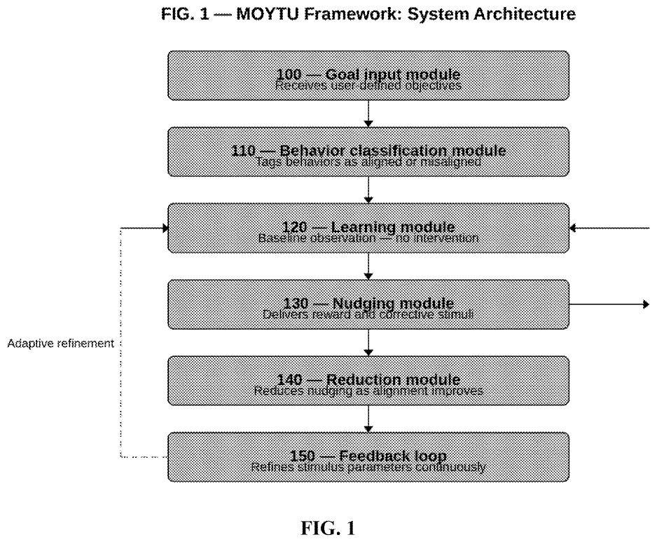

FIG. 2 is a flowchart illustrating the three sequential operational phases of the MOYTU Framework, comprising the goal-setting phase, the baseline learning period, and the active nudging period, including the gradual reduction of stimulus frequency as behavioral alignment improves and the system endpoint.

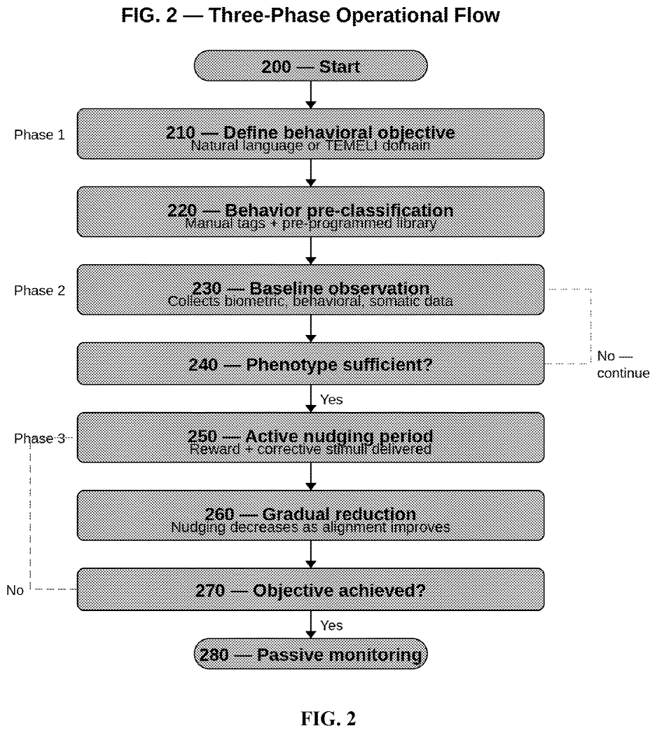

FIG. 3 is a flowchart illustrating the dual-stimulus reinforcement architecture of the MOYTU Framework, showing the detection and classification of user behavior as aligned or misaligned, the selection and delivery of reward or corrective stimuli, the measurement of physiological and behavioral response, and the adaptive feedback loop.

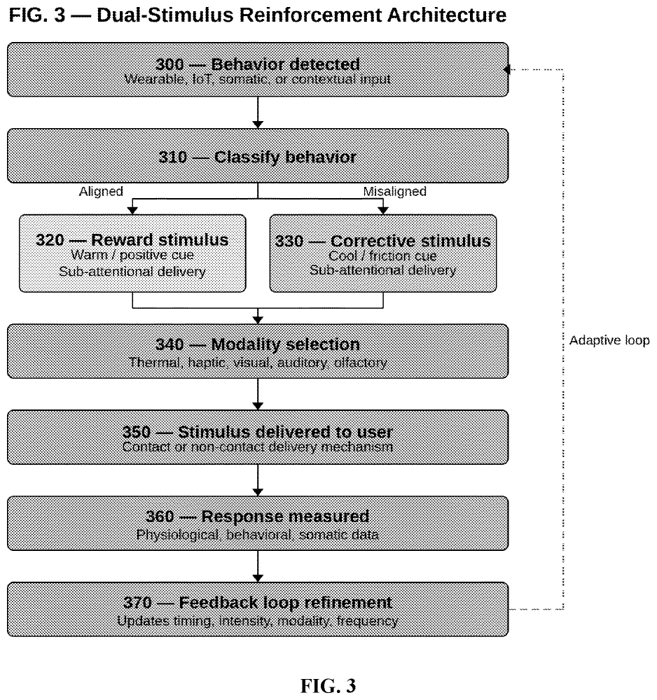

FIG. 4 is a diagram illustrating the TEMELI Framework as integrated within the MOYTU goal input module, showing the six foundational behavioral domains: Breath, Sleep, Movement, Nutrition, Mindfulness, and Prevention, and their corresponding pre-programmed behavioral alignment targets available for user goal selection.

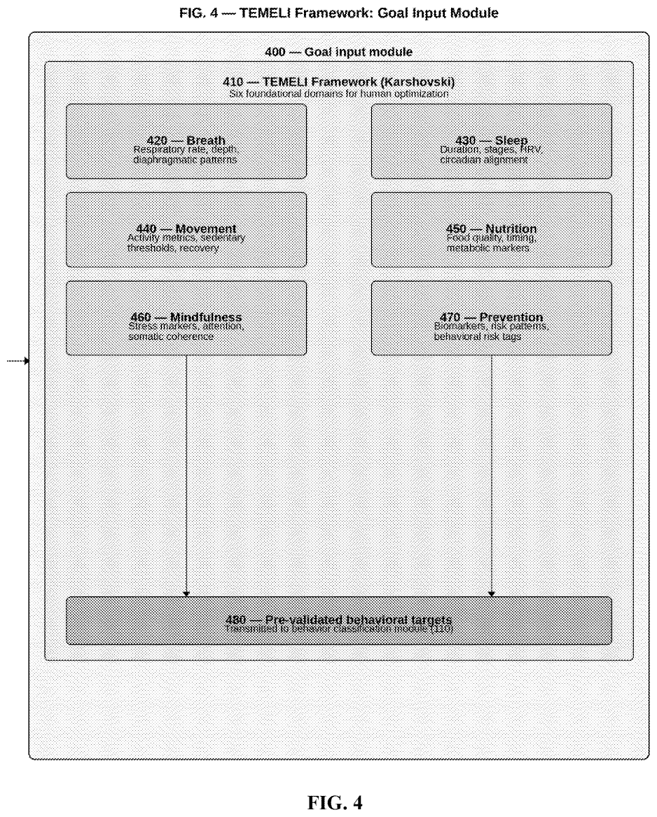

FIG. 5 is a structural diagram illustrating the somatic intelligence layer of the MOYTU Framework, showing the integration of user-reported subjective somatic assessments, interoceptive signals, and autonomic nervous system outputs as first-class inputs to the behavior classification module, and the somatic intelligence mapping module's longitudinal correlation function.

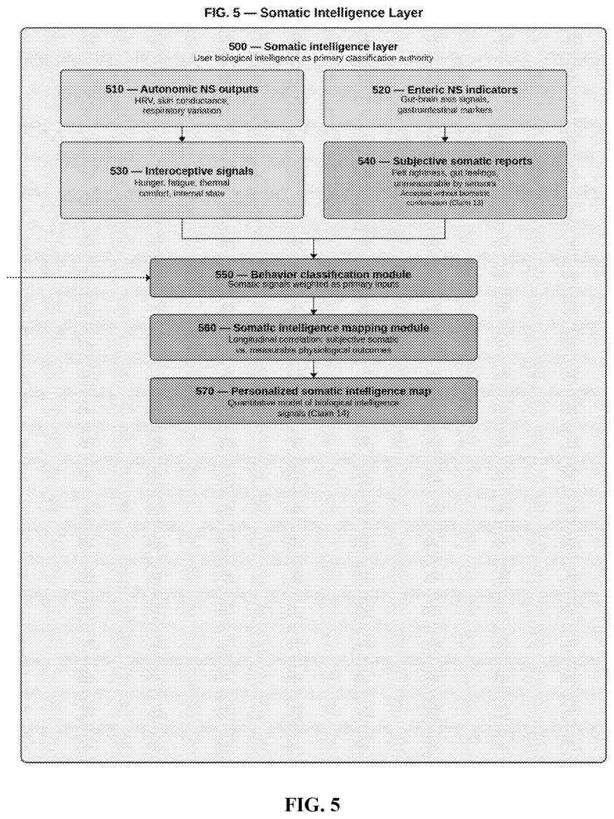

### DETAILED DESCRIPTION OF THE INVENTION

### A. Overview and System Architecture

The MOYTU Framework is a system for goal-directed behavioral optimization that closes the gap between human intention and human action. The system operates through six interconnected modules: a goal input module, a behavior classification module, a learning module, a nudging module, a reduction module, and a feedback loop. These modules operate sequentially across three phases—the goal-setting phase, the baseline learning period, and the active nudging period—and collectively implement a closed-loop behavioral execution system that operates without requiring continuous conscious participation from the user.

The system is implementable via any information-processing architecture, including but not limited to digital computing systems, machine learning models, artificial neural networks, quantum computing systems, biological computing substrates, or any hybrid combination thereof. The system interfaces with wearable sensing devices, environmental sensors, IoT-connected devices, and user-input interfaces to collect multimodal data about the user's physiological, behavioral, contextual, and somatic state.

The system is designed to resist the habituation effects that defeat all existing signal-based behavioral systems by dynamically varying stimulus timing, modality, intensity, and emotional tone. The system's goal is its own progressive obsolescence in each behavioral domain—embedding new behavioral patterns so durably that external reinforcement becomes unnecessary.

### B. Goal Input Module

The goal input module is the entry point of the MOYTU Framework. It receives one or more user-defined behavioral or lifestyle objectives and translates them into operational parameters that govern the subsequent behavior of all downstream modules.

User-defined objectives may be expressed in natural language—for example, “help me sleep better,” “reduce my sugar consumption,” or “increase my daily movement”13 and are processed by the system into structured behavioral targets. The goal input module supports three modes of objective definition.

In the first mode, the user freely defines a custom behavioral objective in their own terms. The system parses the objective and maps it to measurable behavioral and physiological indicators that serve as alignment criteria throughout the system's operation.

In the second mode, the user selects from a pre-structured library of objectives organized according to the TEMELI Framework—a holistic behavioral health system developed by the inventor, Doychin Karshovski. The TEMELI Framework organizes human optimization across six foundational domains: Breath, Sleep, Movement, Nutrition, Mindfulness, and Prevention. Each domain contains pre-validated behavioral targets with established relationships to measurable health outcomes. These pre-structured objectives reduce the goal-definition burden on the user and ensure that selected goals are grounded in evidence-based health science. The six TEMELI domains serve as the primary pre-programmed goal library of the MOYTU system and provide the philosophical and structural backbone against which behavioral alignment is assessed.

In the third mode, the user combines both approaches—selecting a TEMELI domain as a primary orientation and customizing the specific behavioral targets within that domain according to their personal circumstances, preferences, and somatic intelligence.

The goal input module further accepts user-initiated manual tagging of specific behaviors as aligned or misaligned with the stated objective, prior to or during the learning period. This allows users to contribute their own behavioral knowledge and somatic awareness to the classification process from the outset, rather than waiting for the system to derive classifications through automated correlation alone.

The goal input module also interfaces with a pre-programmed behavioral library containing behaviors classified as misaligned by default based on established health outcome data. These pre-classified behaviors—including but not limited to smoking, prolonged sedentary behavior, chronic sleep disruption, and habitual consumption of nutritionally depleted foods—do not require a learning period to classify and may trigger corrective stimulus delivery immediately upon detection.

Upon completion of goal definition, the goal input module transmits the structured objective parameters, behavioral alignment criteria, and any user-defined or pre-programmed behavioral classifications to the behavior classification module and the learning module, initiating the baseline observation period.

### C. Behavior Classification Module

The behavior classification module receives structured objective parameters from the goal input module and continuously evaluates observed user behaviors against those parameters, assigning each behavior a classification of aligned or misaligned with the stated objective.

Classification is performed through three concurrent mechanisms that operate independently and in combination.

The first mechanism is automated correlation analysis. The behavior classification module receives behavioral, biometric, contextual, and somatic data streams from the learning module and applies pattern recognition to identify statistical correlations between observed behaviors and measured outcomes related to the stated objective. Behaviors that correlate positively with improved objective outcomes are classified as aligned. Behaviors that correlate negatively—or that precede measurable deterioration in objective-relevant outcomes—are classified as misaligned. This automated classification process operates continuously and updates classifications dynamically as new data is collected.

The second mechanism is user-initiated manual tagging. At any point during system operation, the user may designate specific behaviors as aligned or misaligned based on their own direct experience, somatic awareness, or personal knowledge. These user-initiated classifications are treated as first-class data inputs and are weighted alongside automated classifications in the system's behavioral model. This mechanism explicitly incorporates the user's innate biological intelligence—including subjective felt states of rightness, intuitive assessments of wellbeing, and somatic coherence experiences that are not currently measurable by external sensing technologies—as valid and authoritative classification data. The system does not require objective biometric confirmation to accept and act on user-reported somatic assessments.

The third mechanism is pre-programmed behavioral library retrieval. The behavior classification module maintains a library of behaviors with established health outcome relationships, pre-classified as misaligned by default. These include but are not limited to: smoking, prolonged sedentary behavior exceeding defined thresholds, chronic sleep disruption, habitual consumption of nutritionally depleted foods, and other behaviors with documented negative health outcome correlations. Behaviors in this library do not require a learning period and may be classified and acted upon immediately upon detection. Pre-programmed classifications may be supplemented or overridden by user-initiated manual tagging, allowing individual variation and personal somatic intelligence to take precedence over population-level defaults where appropriate.

The behavior classification module further incorporates an interoceptive signal processing layer. This layer receives and processes autonomic nervous system outputs, enteric nervous system indicators, and user-reported somatic awareness signals as inputs to the classification function. Behaviors producing consistent positive autonomic and physiological responses—even in the absence of externally defined health outcome data confirming their benefit—may be classified as aligned based on somatic coherence signals alone. This mechanism positions the user's own biological intelligence as a primary classification authority, not merely a supplementary data source.

All three classification mechanisms feed into a unified behavioral alignment model that is continuously updated throughout system operation. The output of the behavior classification module—a dynamic, personalized map of the user's aligned and misaligned behaviors—is transmitted to the learning module during the observation period and to the nudging module upon initiation of the active nudging phase.

### D. Learning Module

The learning module governs the baseline observation period—the second phase of MOYTU Framework operation. During this phase, the system collects multimodal data about the user's physiological, behavioral, contextual, and somatic state without delivering any stimuli or interventions. The learning module operates silently. The user experiences no active system output during this period.

### The Learning Module Collects Data From Four Primary Input Streams.

The first stream is physiological data, received from wearable and proximate sensing devices. This includes but is not limited to heart rate variability, resting heart rate, sleep stages and duration, respiratory rate, skin temperature, and physical activity metrics including steps, movement intensity, and sedentary period duration. The learning module is further configured to integrate real-time biochemical and neurological data streams as such sensing technologies become available, including blood-based biomarkers, urinary metabolites, neurochemical indicators, and any measurable biological output accessible through non-invasive, minimally invasive, or future sensing technologies not yet in existence at the time of filing.

The second stream is contextual data, collected from environmental sensors, device usage logs, and location services. This includes time of day, day of week, geolocation context, ambient light levels, ambient sound levels, screen time, application usage patterns, and digital behavioral markers such as scrolling patterns, screen unlock frequency, and food delivery application usage.

The third stream is behavioral marker data, derived from IoT-connected devices and digital environment monitoring. This includes data from smart home devices, kitchen appliances, connected fitness equipment, and any other device capable of reporting behavioral events relevant to the stated objective.

The fourth stream is user-reported somatic input. As described in Section C, the learning module accepts user-reported subjective somatic assessments—including felt states of rightness, intuitive assessments of wellbeing, and somatic coherence experiences not currently measurable by external sensing technologies—as first-class data inputs throughout the observation period.

Using these four data streams, the learning module constructs a personalized behavioral phenotype—a dynamic, continuously updated model of the user's unique behavioral patterns, physiological rhythms, contextual triggers, somatic responses, and temporal habits. This phenotype serves as the personalized baseline against which all subsequent nudging decisions are calibrated.

The learning module continuously transmits updated behavioral classifications to the behavior classification module throughout the observation period. When the behavioral phenotype reaches sufficient resolution to support reliable nudge targeting—as determined by the system's adaptive algorithms or by a pre-programmed threshold defined at goal setup—the learning module signals the nudging module to initiate the active nudging phase.

The duration of the learning period is variable and user-specific. It is determined by the complexity of the stated objective, the richness of available data streams, and the speed at which reliable behavioral correlations emerge. The learning period does not have a fixed endpoint. It may be pre-programmed with a minimum duration or left to adaptive determination by the system.

### E. Nudging Module and Dual-stimulus Architecture

The nudging module governs the active nudging period—the third phase of MOYTU Framework operation. It is initiated upon signal from the learning module that the behavioral phenotype has reached sufficient resolution to support reliable intervention. The nudging module is responsible for selecting, timing, calibrating, and delivering stimuli to the user in a manner that promotes behavioral alignment with the stated objective without requiring conscious interpretation, decision, or volitional effort from the user.

The nudging module employs a dual-stimulus architecture modeled on the fundamental principles of operant conditioning and behavioral reinforcement. Two categories of stimulus are available to the system.

The first category is reward stimuli. Reward stimuli are delivered when the user engages in a behavior classified as aligned with the stated objective. Reward stimuli are calibrated to produce a subtle positive physiological or somatic response—a felt sense of ease, pleasure, calm, or vitality—that the user's nervous system associates with the preceding aligned behavior. Over repeated delivery, this association progressively strengthens the user's sub-attentional preference for aligned behaviors without requiring conscious awareness of the reinforcement mechanism.

The second category is corrective stimuli. Corrective stimuli are delivered when a behavior classified as misaligned with the stated objective is detected or predicted by the system. Corrective stimuli are calibrated to produce a subtle negative physiological or somatic response—a felt sense of discomfort, unease, resistance, or friction—that the user's nervous system associates with the preceding or anticipated misaligned behavior. Over repeated delivery, this association progressively reduces the user's sub-attentional preference for misaligned behaviors.

This dual-stimulus architecture—reward for aligned behavior, correction for misaligned behavior, both operating below the threshold of active conscious resistance—constitutes the core behavioral execution mechanism of the MOYTU Framework.

Stimuli are delivered through any contact or non-contact mechanism capable of producing the designated physiological or somatic response in the user, including but not limited to direct skin-contact devices, proximate field-based delivery systems, infrared or radiant energy systems, ultrasonic or acoustic field systems, electromagnetic systems, or any future delivery mechanism not yet in existence at the time of filing.

Current stimulus modalities include but are not limited to: thermal signals such as subtle warm and cool temperature variations delivered through any contact or non-contact mechanism capable of producing warm or cool somatic sensations in the user—including direct skin-contact thermal devices, infrared radiant systems, or any future thermal delivery technology—wherein warm thermal stimuli are preferentially associated with reward states and cool thermal stimuli are preferentially associated with corrective states, thereby encoding behavioral reinforcement meaning through temperature polarity below the threshold of conscious resistance; haptic signals such as vibration patterns of varying frequency, duration, and intensity delivered through any body-contact or proximate device; visual cues such as ambient light color shifts, screen tint adjustments, luminosity changes, or notification-free interface modifications; auditory prompts such as tones, breath rhythm cues, binaural audio patterns, or subliminal audio delivered through connected audio devices; olfactory signals delivered through connected ambient scent devices or any future olfactory delivery mechanism; and any other stimulus modality capable of influencing user behavior through any sensory, physiological, or neurological pathway, whether currently known or developed after the time of filing.

The nudging module is further configured to deliver stimuli in coordinated multi-modal combinations, wherein two or more stimulus modalities are delivered simultaneously or in calibrated temporal sequence to produce a compound behavioral signal. Multi-modal combinations are selected and calibrated by the contextual intelligence layer to maximize sub-attentional efficacy, minimize habituation, and encode more precise behavioral meaning than any single modality alone is capable of delivering. The invention explicitly claims all combinations of two or more stimulus modalities delivered in coordination for the purpose of behavioral reinforcement, whether such combinations are currently implemented or developed after the time of filing.

The nudging module further supports deliberate stimulus-to-tag assignment, wherein specific stimulus modalities are permanently or dynamically assigned to specific behavioral classification categories. In a preferred implementation, a designated reward modality—such as a warm thermal signal—is consistently delivered in response to aligned behavior detections, while a designated corrective modality—such as a cool thermal signal, a distinct haptic pattern, or a specific visual cue—is consistently delivered in response to misaligned behavior detections. Through repeated consistent pairing of modality with behavioral classification, the system establishes a sub-attentional stimulus-encoding framework in which the user's nervous system learns the behavioral meaning of each stimulus modality without conscious instruction.

Stimulus selection and calibration are governed by the contextual intelligence layer of the nudging module, which evaluates the user's current physiological state, environmental context, time of day, recent stimulus history, and measured responsiveness to prior stimuli of each modality. The nudging module further incorporates an emotional timing filter that identifies optimal intervention windows and withholds stimulus delivery during moments of acute stress, emotional distress, or physiological crisis.

In an extended implementation, the nudging module may be embodied within a physically autonomous agent operating in the user's environment—including but not limited to humanoid robotic systems, mobile robotic platforms, or any other physically embodied artificial agent. The embodied agent serves as the primary stimulus delivery system, observing user behavior through onboard sensors and delivering reward and corrective stimuli through the full repertoire of physical interaction modalities available to an embodied agent—including vocal communication, gestural interaction, environmental manipulation, and physical proximity variation. The embodied agent operates within a stimulus repertoire and intensity range pre-approved by the user at goal setup.

In its most advanced implementation, the nudging module delivers stimuli directly through non-invasive neural interface technologies capable of modulating cognitive states, emotional valence, attentional focus, or behavioral impulse strength at the neurological level. Current technologies include transcranial electrical and magnetic stimulation devices, focused ultrasound neural modulation systems, and non-invasive electroencephalographic feedback systems. All such technologies, whether currently available or developed after the time of filing, are explicitly included within the MOYTU Framework's nudging module implementation scope, provided they operate without permanent biological modification and within a stimulus repertoire pre-approved by the user.

### F. Reduction Module and System Endpoint

The reduction module governs the progressive decrease of stimulus delivery frequency as behavioral alignment with the stated objective improves. Unlike systems that maintain or increase intervention intensity over time, the MOYTU Framework is designed with the explicit goal of its own progressive obsolescence in each behavioral domain.

The reduction module continuously monitors behavioral alignment metrics and reduces nudging frequency in proportion to measured improvement. This reduction is gradual and non-linear—calibrated to maintain behavioral momentum while progressively withdrawing external reinforcement as internal behavioral preferences shift toward alignment.

The system endpoint may be defined in two ways. In the first mode, the endpoint is pre-programmed at goal setup, defined by a threshold level of behavioral alignment maintained over a specified duration. In the second mode, the endpoint is user-defined. Upon reaching the system endpoint, the learning module continues passive monitoring without delivering stimuli. If behavioral misalignment re-emerges above a defined threshold after goal completion, the system may automatically re-initiate a modified nudging protocol or alert the user to the detected regression.

The reduction module operates independently for each active objective, allowing simultaneous management of multiple behavioral goals at different stages of the three-phase process.

### G. Feedback Loop and Adaptive Refinement

The feedback loop is the adaptive intelligence layer of the MOYTU Framework. It continuously monitors physiological, behavioral, and somatic responses produced by each stimulus delivery event and uses those measurements to refine all parameters governing future stimulus selection, timing, calibration, and delivery.

Following each stimulus delivery event, the feedback loop collects response data across four dimensions. The first dimension is physiological response—measurable changes in biometric indicators occurring within a defined post-stimulus window. Physiological response data further includes real-time biochemical markers accessible through emerging and future sensing technologies. Current implementations include continuous glucose monitoring systems capable of detecting blood sugar fluctuations in real time—providing direct metabolic feedback correlating behavioral events with immediate physiological outcomes. Near-term implementations include sweat-based biosensing through smart fabric technologies capable of detecting cortisol, lactate, electrolytes, and other metabolic markers through perspiration without any active user action. Future implementations include urine-based metabolite sensing, blood-based continuous biomarker monitoring, and any additional biochemical feedback stream accessible through non-invasive, minimally invasive, or contact-free sensing technologies not yet in existence at the time of filing.

The second dimension is behavioral response—observable changes in user behavior following stimulus delivery. The third dimension is somatic response—user-reported subjective assessments of how a stimulus felt. The fourth dimension is longitudinal outcome correlation—the relationship between cumulative stimulus delivery patterns and measurable progress toward the stated objective over time.

Using these four response dimensions, the feedback loop continuously updates seven stimulus parameters: timing, modality selection, intensity, duration, frequency, emotional tone, and inter-stimulus interval—varied systematically to prevent habituation.

The feedback loop further operates the somatic intelligence mapping function, which longitudinally correlates user-reported subjective somatic signals with subsequently measurable physiological and behavioral outcomes. Over time, the system builds a personalized somatic intelligence map—a dataset that quantifies the predictive relationship between this user's subjective felt sense and their objective physiological reality. This map contributes to the broader dataset described in Claim 14 for the quantitative modeling of biological intelligence signals.

### H. Somatic Intelligence Layer

The somatic intelligence layer is a distinct functional component of the MOYTU Framework that positions the user's own biological intelligence as a primary authority in the system's behavioral classification and refinement processes.

The somatic intelligence layer is founded on the recognition that the human body possesses information-processing capabilities not fully captured by currently measurable biometric indicators. The enteric nervous system—comprising approximately 500 million neurons distributed throughout the gastrointestinal tract—processes information and generates signals that influence behavior independently of conscious brain activity. The autonomic nervous system continuously monitors and responds to internal and external stimuli below the threshold of conscious awareness, generating what are commonly described as gut feelings, intuitive responses, or somatic felt senses.

The somatic intelligence layer accepts four categories of input: autonomic nervous system output data; enteric nervous system indicators; interoceptive signal data representing the body's internal sense of its own physiological state; and user-reported subjective somatic assessments. User-reported subjective somatic assessments include felt states of rightness or wrongness regarding specific behaviors, intuitive assessments of wellbeing following behavioral events, and somatic coherence experiences indicating alignment between behavior and biological preference. These assessments are accepted as valid first-class behavioral alignment data independently of and without requiring confirmation from objective biometric measurements.

The somatic intelligence mapping module performs longitudinal correlation analysis between user-reported subjective somatic signals and subsequently measurable physiological and behavioral outcomes. Over time, as patterns accumulate, the module builds a personalized somatic intelligence map—a quantitative model of the relationship between this user's subjective felt sense and their objective physiological reality. The aggregated, anonymized somatic intelligence data generated across the MOYTU user population contributes to a growing dataset correlating subjective somatic experience with measurable biological outcomes—representing a novel contribution to the scientific understanding of human biological intelligence.

### I. Optional Suggestive Priming Module

The MOYTU Framework optionally includes a suggestive priming module that enhances the efficacy of subsequently delivered nudging stimuli by establishing sub-attentional associative pathways prior to the active nudging phase.

The suggestive priming module operates on the principle of conditioned stimulus association. A neutral stimulus that will subsequently be used as a nudging stimulus is deliberately paired with a desired emotional or behavioral state during a guided priming session. Through repeated pairing, the stimulus acquires associative meaning at the subconscious level. When subsequently delivered by the nudging module, it carries pre-loaded behavioral meaning—triggering the associated state without requiring conscious interpretation.

Priming sessions are delivered through guided protocols that induce heightened neurological receptivity. Current protocols include hypnosis, deep meditation, visualization, and breath-based relaxation techniques. The suggestive priming module interfaces with the somatic intelligence layer to identify moments of peak neurological receptivity during priming sessions, delivering stimulus-state pairings preferentially during these windows.

In advanced implementations incorporating non-invasive neural interface technologies, the suggestive priming module may deliver priming stimuli directly at the neurological level—bypassing the need for guided relaxation protocols by directly inducing the neurological state associated with heightened receptivity.

Priming may occur at onboarding or periodically during the active nudging phase to refresh and deepen conditioned associations. The system tracks associative pathway strength over time and recommends priming session renewal when efficacy falls below a defined threshold.

### J. Institutional Deployment

The MOYTU Framework is designed for deployment across institutional contexts in addition to individual consumer use. Institutional deployment enables the system to operate across populations of users simultaneously—with each user maintaining an independent, personalized behavioral profile—while aggregate anonymized behavioral data contributes to population-level refinement of classification and nudging parameters.

Institutional deployment contexts include corporate wellness environments, military and security contexts, clinical and medical settings, and educational environments.

In corporate wellness environments, the system supports large-scale employee behavioral health programs without requiring active employee participation beyond initial goal setup. In military and security contexts, the system supports high-performance behavioral conditioning and may be configured for secure offline deployment through wearable devices operating without continuous network connectivity. In clinical and medical settings, the system supports long-term behavioral adherence to treatment protocols—including recovery from addiction, management of chronic health conditions, and physiotherapy compliance—by delivering continuous sub-attentional reinforcement without requiring therapist presence between appointments. In educational environments, the system supports attention regulation and healthy habit formation in children and adolescents through age-appropriate stimulus modalities calibrated to developmental stage.

In all institutional deployments, individual user data is maintained with strict privacy separation. Aggregate behavioral data is anonymized prior to processing. Ethical deployment guidelines—including user informed consent, stimulus repertoire transparency, and user override capability—are maintained across all institutional implementations.

### K. Example Use Cases

The following example use cases illustrate the operation of the MOYTU Framework across representative deployment contexts. These examples are provided for illustrative purposes and do not limit the scope of the invention as defined by the claims.

### Example 1—Individual Consumer: Sleep Optimization

A 44-year-old corporate professional defines the goal “help me sleep better” through the goal input module. The system maps this objective to the Sleep domain of the TEMELI Framework and activates the baseline learning period. Over a 14-day observation period, the learning module collects HRV data, sleep stage data, screen time logs, caffeine consumption patterns, and subjective somatic reports—including a notation that “a glass of wine after dinner feels relaxing but wrong.” This user-reported somatic assessment is accepted as a first-class misaligned behavior indicator without requiring biometric confirmation.

The behavior classification module identifies three consistently misaligned behaviors: screen use after 21:00, caffeine consumption after 15:00, and alcohol consumption within three hours of sleep. Upon completion of the learning period, the nudging module initiates the active phase. At 20:45 each evening, a warm thermal signal is delivered—reward stimulus for the aligned evening wind-down window. At 21:00, if screen use is detected, a cool thermal stimulus follows—corrective cue delivered before the user has consciously decided to continue scrolling. No notification appears. No action is requested. Over eleven weeks, the user's average sleep duration increases by 47 minutes. The user reports noticing one day that they simply stopped reaching for their phone after dinner—without having consciously decided to.

### Example 2—Clinical Setting: Addiction Recovery

A patient in an outpatient addiction recovery program defines the goal “reduce my impulse to use” through a clinician-configured institutional deployment. The pre-programmed behavioral library immediately classifies substance use behaviors as misaligned without requiring a learning period. The learning module monitors HRV patterns to identify physiological pre-patterns that reliably precede craving states for this individual.

Upon detection of a pre-craving HRV pattern, the nudging module delivers a coordinated multi-modal corrective stimulus—a cool thermal signal combined with a subtle auditory tone—calibrated to interrupt pre-conscious craving formation before it reaches conscious awareness. A warm thermal reward stimulus is delivered whenever the user engages in aligned behaviors such as calling a support contact or completing a breathing exercise. The suggestive priming module was activated at onboarding—during a guided meditation session, specific haptic patterns were pre-associated with states of calm and self-efficacy. Twelve weeks into the program, the patient's self-reported craving frequency has decreased by 60%.

### Example 3—Institutional: Military Performance Conditioning

A special operations unit deploys the MOYTU Framework through secure offline-capable wearable devices. Personnel define individual performance goals within a command-configured institutional behavioral library covering sleep optimization, hydration, movement recovery, and cognitive readiness.

During high-stress operational periods, the learning module detects HRV degradation patterns associated with decision fatigue. The nudging module delivers a brief coordinated thermal and haptic stimulus sequence pre-associated during onboarding with a state of focused readiness. Personnel report no conscious awareness of the intervention but demonstrate measurably improved decision accuracy scores in post-operation assessments. Sleep recovery nudges are delivered through ambient light modulation in sleeping quarters—blue-shifted light during wake periods transitioning to warm amber tones during sleep onset windows.

### Example 4—Advanced Implementation: Humanoid Agent and Neural Interface

In a near-future implementation, a user deploys the MOYTU Framework through a combination of a wearable biometric device, a home-deployed humanoid robotic agent, and a non-invasive neural wristband. The user has defined goals across three TEMELI domains: movement, nutrition, and mindfulness.

When the learning module detects a pre-pattern associated with impulsive food consumption—a specific sequence of kitchen proximity, elevated cortisol detected through a smart fabric interface, and time-of-day context—the humanoid agent delivers a gentle environmental intervention: it dims the kitchen lighting slightly and moves pre-approved foods to a more prominent position, while the neural wristband delivers a sub-threshold transcranial stimulus associated during priming with the felt sense of satiety and calm. No verbal instruction is given. No notification appears. The user reaches for the pre-approved food without conscious awareness of the intervention. The somatic intelligence mapping module logs the user's subsequent self-report—“felt satisfied, didn't want the other thing”—correlating it with the cortisol reduction measured 20 minutes later, contributing to the user's personalized somatic intelligence map.

What is claimed is:

1. A system for goal-directed behavioral optimization, implemented via any information-processing architecture, comprising:
a goal input module configured to receive one or more user-defined behavioral objectives;
a behavior classification module configured to tag observed behaviors as aligned or misaligned with said objectives through one or more of: automated correlation analysis of collected data, user-initiated manual classification, or retrieval from a pre-programmed library of behaviors with established health outcomes;
a learning module configured to collect biometric, behavioral, somatic, and outcome data over a baseline observation period prior to any active intervention, and to refine behavior classifications based on detected patterns;
a nudging module configured to deliver low-friction stimuli through any sensory, physiological, or neurological pathway available to the user, wherein said stimuli are calibrated to promote behavioral alignment with stated objectives without requiring active conscious effort from the user;
a reduction module configured to decrease stimulus frequency proportionally as behavioral alignment with stated objectives improves; and
a feedback loop configured to continuously refine stimulus timing, pathway, and intensity based on measured and reported behavioral, physiological, and somatic outcomes.

2. A method for goal-directed behavioral optimization, executable via any information-processing architecture, comprising:
receiving one or more user-defined behavioral objectives;
collecting biometric, behavioral, somatic, and outcome data from a user over a baseline observation period;
classifying observed behaviors as aligned or misaligned with said objectives through automated analysis, user-initiated manual input, pre-programmed behavioral libraries, or any combination thereof;
delivering low-friction stimuli through any sensory, physiological, or neurological pathway available to the user upon completion of the baseline observation period, wherein said stimuli promote behavioral alignment without requiring active conscious effort;
reducing stimulus frequency proportionally as behavioral alignment improves; and
continuously refining stimulus parameters based on measured and reported outcomes.

3. The system of claim 1, wherein said stimuli are delivered through one or more modalities selected from haptic signals, visual cues, auditory prompts, olfactory signals, thermal signals, or any combination thereof.

4. The system of claim 1, wherein said stimuli are further calibrated to operate at or below the threshold of conscious perception, such that behavioral influence occurs without the user's active awareness of the intervention.

5. The system of claim 1, wherein the goal input module is further configured to align user-defined objectives with one or more of six foundational behavioral domains comprising breath, sleep, movement, nutrition, mindfulness, and prevention, and wherein the behavior classification module applies domain-specific behavioral criteria corresponding to said alignment.

6. The system of claim 1, wherein the behavior classification module comprises a pre-programmed behavioral library containing behaviors classified as misaligned by default based on established health outcome data, including but not limited to smoking, prolonged sedentary behavior, and chronic sleep disruption, and wherein said pre-programmed classifications may be supplemented or overridden by user-initiated manual tagging.

7. The system of claim 1, wherein the learning module is configured to collect biometric data comprising one or more of heart rate variability, resting heart rate, sleep stages and duration, respiratory rate, skin temperature, physical activity metrics, and blood-based physiological markers accessible through wearable or proximate sensing devices.

8. The system of claim 1, wherein the learning module is further configured to integrate real-time biochemical and neurological data streams including but not limited to blood-based biomarkers, urinary metabolites, neurochemical indicators, interoceptive signals, autonomic nervous system outputs, enteric nervous system indicators, and any measurable biological output accessible through non-invasive, minimally invasive, or future sensing technologies not yet in existence at the time of filing.

9. The system of claim 1, optionally comprising a suggestive priming module configured to pre-associate one or more sensory stimuli with desired behavioral or emotional responses through guided protocols including hypnosis, visualization, meditation, or other cognitive conditioning techniques, thereby enhancing the efficacy of subsequently delivered nudging stimuli.

10. The system of claim 1, wherein the system is further configured for deployment across multiple users within an institutional setting, wherein individual behavioral profiles are maintained independently while aggregate anonymized behavioral data is used to refine classification and nudging parameters at population scale, applicable to settings including but not limited to military, clinical, educational, and corporate wellness environments.

11. The system of claim 1, wherein the learning module is further configured to receive and process interoceptive signals representing the user's internal physiological state, including autonomic nervous system outputs, enteric nervous system indicators, and subjective self-reported somatic awareness, and wherein said signals are incorporated as inputs to the behavior classification module independently of or in combination with externally measured biometric data.

12. The system of claim 1, wherein the behavior classification module is further configured to validate behavioral alignment through somatic coherence signals, wherein behaviors producing consistent positive autonomic and physiological responses are classified as aligned independently of externally defined health outcome datasets, thereby incorporating the user's innate biological intelligence as a primary classification authority.

13. The system of claim 1, wherein the behavior classification module is further configured to accept user-reported subjective somatic assessments as valid behavioral alignment indicators, including felt states of rightness, intuitive assessments of wellbeing, and somatic coherence experiences that are not currently measurable by any external sensing technology, wherein said subjective reports are treated as first-class data inputs independently of objective biometric confirmation.

14. The system of claim 1, further comprising a somatic intelligence mapping module configured to longitudinally correlate user-reported subjective somatic signals with subsequently measurable physiological and behavioral outcomes, thereby generating datasets that contribute to the quantitative modeling of biological intelligence signals not currently measurable at the time of user reporting.

15. The system of claim 1, wherein thermal stimuli are delivered through any contact or non-contact mechanism capable of producing warm or cool somatic sensations in the user, including but not limited to direct skin-contact thermal devices, infrared radiant systems, or any future thermal delivery technology, wherein warm thermal stimuli are preferentially calibrated for delivery as reward cues and cool thermal stimuli are preferentially calibrated for delivery as corrective cues, thereby encoding behavioral reinforcement polarity through temperature variation independently of delivery mechanism or anatomical location.

16. The system of claim 1, wherein the nudging module is configured to deliver coordinated multi-modal stimulus combinations comprising two or more stimulus modalities delivered simultaneously or in calibrated temporal sequence through any contact or non-contact delivery mechanism, wherein said combinations are selected to maximize sub-attentional behavioral efficacy, minimize habituation response, and encode behavioral reinforcement meaning with greater precision than single-modality delivery, including but not limited to combinations of thermal, haptic, visual, auditory, and olfactory stimuli in any configuration and through any delivery mechanism whether currently known or developed after the time of filing.

17. The system of claim 1, wherein the nudging module is configured to assign specific stimulus modalities to specific behavioral classification categories, such that a designated modality is consistently delivered as a reward cue for aligned behaviors and a different designated modality is consistently delivered as a corrective cue for misaligned behaviors, thereby establishing a personalized sub-attentional stimulus-to-behavior encoding system wherein the user's nervous system learns to associate each modality with its corresponding behavioral meaning without conscious instruction or awareness.

18. The system of claim 1, wherein the nudging module is embodied in a physically autonomous agent—including but not limited to a humanoid robot, a mobile robotic system, or any other physically embodied artificial agent capable of operating within the user's environment—wherein said agent observes user behavior through onboard sensing systems, delivers reward and corrective stimuli through physical interaction, environmental manipulation, vocal cues, gestural communication, or any other mechanism available to a physically embodied system, and operates within a repertoire of stimulus types and intensities pre-approved by the user at goal setup, such that the physically embodied agent functions as an autonomous behavioral execution partner operating on behalf of the user's stated objectives without requiring ongoing conscious direction from the user.

19. The system of claim 1, wherein the nudging module delivers stimuli directly through neural interface technologies—including but not limited to non-invasive transcranial stimulation, focused ultrasound neural modulation, electromagnetic neural field stimulation, or any future non-invasive or minimally invasive technology capable of directly influencing neural activity, cognitive states, emotional valence, or behavioral impulses—wherein said neural stimuli are calibrated to promote behavioral alignment with user-defined objectives by modulating the user's neurological state below the threshold of conscious resistance, without requiring surgical intervention or permanent biological modification.
